(19) United States
(12) Patent Application Publication
(10) Pub. No.: US 2019/0125772 A1
(43) Pub. Date: May 2, 2019

(54) IMPROVED DRUG COMBINATIONS FOR DRUG-RESISTANT AND DRUG-SENSITIVE MULTIPLE MYELOMA

(71) Applicants: THE REGENTS OF THE UNIVERSITY OF CALIFORNIA, Oakland, CA (US); NATIONAL UNIVERSITY OF SINGAPORE, Singapore (SG)

(72) Inventors: Dean HO, Los Angeles, CA (US); Chih-Ming HO, Los Angeles, CA (US); Aleidy Marlene Silva VITE, Los Angeles, CA (US); Kangyi ZHANG, Los Angeles, CA (US); Edward Kai-Hua CHOW, Singapore (SG); Tan Boon TOH, Singapore (SG); Wee Joo CHNG, Singapore (SG); Masturah Mohd Abdul RASHID, Singapore (SG)

(73) Assignees: THE REGENTS OF THE UNIVERSITY OF CALIFORNIA, Oakland, CA (US); NATIONAL UNIVERSITY OF SINGAPORE, Singapore (SG)

(21) Appl. No.: 15/571,457

(22) PCT Filed: May 4, 2016
(86) PCT No.: PCT/US2016/030819

§ 371 Date: Nov. 2, 2017

Related U.S. Application Data
(60) Provisional application No. 62/157,348, filed on May 5, 2015.

(51) Int. Cl. A61K 31/69; A61K 31/131; A61K 31/706; A61K 31/407; A61P 35/00; A61K 31/4045; A61K 31/573

(52) U.S. Cl. CPC A61K 31/69; A61K 31/131; A61K 31/706; A61K 31/573; A61P 35/00; A61K 31/4045; A61K 31/407

(57) ABSTRACT

Disclosed are pharmaceutical compositions and methods of treating multiple myeloma by providing a pharmaceutically effective amount of each drug in a combination of drugs.

### CROSS-REFERENCE TO RELATED APPLICATION

This application claims the benefit of U.S. Provisional Application No. 62/157,348, filed on May 5, 2015, the disclosure of which is incorporated herein by reference in its entirety.

### TECHNICAL FIELD

This disclosure generally relates to multi-drug therapies for multiple myeloma.

### BACKGROUND

The use of drug combinations possesses an important advantage over single drug therapy. Monotherapies often lead to disease recurrence and subsequent ineffectiveness of standard treatment due to drug resistance development. Multi-drug therapies are now the standard treatment for multiple diseases, but their development has involved arduous empirical testing. The design of such therapies is quite challenging since the interactions between drugs are not well understood, as the crossover between the affected cellular pathways is quite difficult to comprehend. Furthermore, combining several drugs at different possible concentrations or dosages yields a large testing parametric space, which makes the search of an optimal combination a major challenge. Therefore, there is a need to use a different approach to develop multi-drug therapies.

It is against this background that a need arose to develop the embodiments described in this disclosure.

### SUMMARY OF DISCLOSURE

In certain aspects, some embodiments of this disclosure are directed to a pharmaceutical composition comprising a pharmaceutically effective amount of each drug in a drug combination selected from the group consisting of:
decitabine and mitomycin C;
bortezomib and mechlorethamine hydrochloride;
decitabine and mechlorethamine hydrochloride;
decitabine, mechlorethamine hydrochloride, and mitomycin C;
bortezomib, decitabine, and mitomycin C;
bortezomib, mechlorethamine hydrochloride, and decitabine; and
bortezomib, mechlorethamine hydrochloride, decitabine, and mitomycin C.
   In some embodiments, the drug combination comprises, or alternatively consists essentially of, or yet further consists of decitabine and mitomycin C. In some embodiments, the drug combination comprises, or alternatively consists essentially of, or yet further consists of bortezomib and mechlorethamine hydrochloride. In some embodiments, the drug combination comprises, or alternatively consists essentially of, or yet further consists of decitabine and mechlorethamine hydrochloride. In some embodiments, the drug combination comprises, or alternatively consists essentially of, or yet further consists of decitabine, mechlorethamine hydrochloride, and mitomycin C. In some embodiments, the drug combination comprises, or alternatively consists essentially of, or yet further consists of bortezomib, decitabine, and mitomycin C. In some embodiments, the drug combination comprises, or alternatively consists essentially of, or yet further consists of bortezomib, mechlorethamine hydrochloride, and decitabine. In some embodiments, the drug combination comprises, or alternatively consists essentially of, or yet further consists of bortezomib, mechlorethamine hydrochloride, decitabine, and mitomycin C. In some embodiments, the pharmaceutically effective amount, or dosage, of each respective drug in the drug combination is below a maximum tolerated dosage of that respective drug. In some embodiments, the pharmaceutical composition consists essentially of, or consists of, the drug combination. In some embodiments, the pharmaceutical composition comprises, or alternatively consists essentially of, or yet further consists of the drug combination and a pharmaceutical acceptable carrier or excipient.

In other aspects, some embodiments of this disclosure are directed to a pharmaceutical composition comprising a pharmaceutically effective amount of each drug in a drug combination selected from the group consisting of:
bortezomib and dexamethasone;
bortezomib, panobinostat, and dexamethasone;
bortezomib, mechloroethamine hydrochloride, and dexamethasone; and
bortezomib, mechloroethamine hydrochloride, panobinostat, and dexamethasone.
   In some embodiments, the drug combination comprises, or alternatively consists essentially of, or yet further consists of bortezomib and dexamethasone. In some embodiments, the drug combination comprises, or alternatively consists essentially of, or yet further consists of bortezomib, panobinostat, and dexamethasone. In some embodiments, the drug combination comprises, or alternatively consists essentially of, or yet further consists of bortezomib, mechloroethamine hydrochloride, and dexamethasone. In some embodiments, the drug combination comprises, or alternatively consists essentially of, or yet further consists of bortezomib, mechloroethamine hydrochloride, panobinostat, and dexamethasone. In some embodiments, the pharmaceutically effective amount, or dosage, of each respective drug in the drug combination is below a maximum tolerated dosage of that respective drug. In some embodiments, the pharmaceutical composition consists essentially of, or consists of, the drug combination. In some embodiments, the pharmaceutical composition comprises, or alternatively consists essentially of, or yet further consists of the drug combination and a pharmaceutical acceptable carrier or excipient.

In other aspects, some embodiments of this disclosure are directed to a method of treating bortezomib-resistant multiple myeloma in a subject in need thereof, comprising, or alternatively consisting essentially of, or yet further consisting of administering to the subject a pharmaceutically effective amount of each drug in a drug combination selected from the group consisting of:
decitabine and mitomycin C;
bortezomib and mechlorethamine hydrochloride;
decitabine and mechlorethamine hydrochloride;
decitabine, mechlorethamine hydrochloride, and mitomycin C;
bortezomib, decitabine, and mitomycin C;
bortezomib, mechlorethamine hydrochloride, and decitabine; and
bortezomib, mechlorethamine hydrochloride, decitabine, and mitomycin C.
   In some embodiments, the drug combination comprises, or alternatively consists essentially of, or yet further consists of decitabine and mitomycin C. In some embodiments, the drug combination comprises, or alternatively consists essentially of, or yet further consists of bortezomib and mechlorethamine hydrochloride. In some embodiments, the drug combination comprises, or alternatively consists essentially of, or yet further consists of decitabine and mechlorethamine hydrochloride. In some embodiments, the drug combination comprises, or alternatively consists essentially of, or yet further consists of decitabine, mechlorethamine hydrochloride, and mitomycin C. In some embodiments, the drug combination comprises, or alternatively consists essentially of, or yet further consists of bortezomib, decitabine, and mitomycin C. In some embodiments, the drug combination comprises, or alternatively consists essentially of, or yet further consists of bortezomib, mechlorethamine hydrochloride, and decitabine. In some embodiments, the drug combination comprises, or alternatively consists essentially of, or yet further consists of bortezomib, mechlorethamine hydrochloride, decitabine, and mitomycin C. In some embodiments, the pharmaceutically effective amount, or dosage, of each respective drug in the drug combination is below a maximum tolerated dosage of that respective drug. In some embodiments, two or more drugs in the drug combination are administered sequentially. In some embodiments, two or more drugs in the drug combination are administered concurrently. In some embodiments, the subject is a mammal. In some embodiments, the subject is a human.

In other aspects, some embodiments of this disclosure are directed to a method of treating bortezomib-resistant multiple myeloma in a subject in need thereof, comprising, or alternatively consisting essentially of, or yet further consisting of administering to the subject a pharmaceutically effective amount of each drug in a drug combination comprising bortezomib and at least one additional drug selected from the group consisting of mechlorethamine hydrochloride, decitabine, and mitomycin C. In some embodiments, the pharmaceutically effective amount, or dosage, of each respective drug in the drug combination is below a maximum tolerated dosage of that respective drug. In some embodiments, two or more drugs in the drug combination are administered sequentially. In some embodiments, two or more drugs in the drug combination are administered concurrently. In some embodiments, the subject is a mammal. In some embodiments, the subject is a human.

In other aspects, some embodiments of this disclosure are directed to a method of treating Bortezomib-sensitive multiple myeloma in a subject in need thereof, comprising, or alternatively consisting essentially of, or yet further consisting of administering to the subject a pharmaceutically effective amount of each drug in a drug combination selected from the group consisting of:
bortezomib and dexamethasone;
bortezomib, panobinostat, and dexamethasone;
bortezomib, mechloroethamine hydrochloride, and dexamethasone; and
bortezomib, mechloroethamine hydrochloride, panobinostat, and dexamethasone.
   In some embodiments, the drug combination comprises, or alternatively consists essentially of, or yet further consists of bortezomib and dexamethasone. In some embodiments, the drug combination comprises, or alternatively consists essentially of, or yet further consists of bortezomib, panobinostat, and dexamethasone. In some embodiments, the drug combination comprises, or alternatively consists essentially of, or yet further consists of bortezomib, mechloroethamine hydrochloride, and dexamethasone. In some embodiments, the drug combination comprises, or alternatively consists essentially of, or yet further consists of bortezomib, mechloroethamine hydrochloride, panobinostat, and dexamethasone. In some embodiments, the pharmaceutically effective amount, or dosage, of each respective drug in the drug combination is below a maximum tolerated dosage of that respective drug. In some embodiments, two or more drugs in the drug combination are administered sequentially. In some embodiments, two or more drugs in the drug combination are administered concurrently. In some embodiments, the subject is a mammal. In some embodiments, the subject is a human.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1: Flowchart showing three stages of a Feedback System Control (FSC) platform for optimizing drug combinations.

FIG. 2: Overview of FSC workflow.

FIG. 3: FSC-derived response surface maps reliably identify interactions. (A) Bortezomib and Panobinostat are synergistic in Bortezomib-sensitive RPMI 8226 multiple myeloma cells. (B) Bortezomib and Panobinostat are antagonistic in Bortezomib-resistant RPMI 8226 multiple myeloma cells. (C) Drug interactions are confirmed by experimental data.

FIG. 4: FSC-derived response surface maps identify previously undescribed drug interactions in Bortezomib-resistant multiple myeloma cells. (A) Bortezomib/Mechloroethamine and Decitabine/Mitomycin C are examples of synergistic drug interactions in Bortezomib-resistant RPMI 8226 multiple myeloma cells. (B) Bortezomib/Dexamethasone and Bortezomib/Panobinostat are examples of antagonistic drug interactions in Bortezomib-resistant RPMI 8226 multiple myeloma cells.

FIG. 5: Experimental validation of FSC-derived synergistic drug interactions. Dose-response viability assay confirms synergistic treatment of Bortezomib-resistant RPMI 8226 multiple myeloma cells with Bortezomib and Mechloroethamine or Decitabine and Mitomycin C.

FIG. 6: Top ranked FSC-derived 2-drug (D3, D5) and 3-drug (D4, D6) combinations against Bortezomib-resistant multiple myeloma with corresponding Combination Index compared to an antagonistic, low-ranked combination (D1). Combination Index denotes antagonistic (>1), additive (=1) or synergistic (<1) drug combinations. Cell viability validation of D1-D4 is also shown.

FIG. 7: An excerpt of a list of drug combinations in a first iteration (experimental run). D1 to D14 represent different drugs listed in FIG. 9. “−1” and “1” represent the absence or presence of the respective drug in the drug combination.

FIG. 8: Table showing the design of three iterations. IC20 represents 20% inhibitory concentration, which is the dosage of drug for 20% inhibition of cell growth. The first and second iterations are drug screening experiments to remove unfavorable drug candidates. In the third iteration, the goal is to determine the optimum dosages of the five favorable drugs.

FIG. 9: The list of 14 drugs used for the first iteration. After the first iteration, the drugs as highlighted are removed. After the second iteration, the drugs as highlighted are removed.

FIG. 10: Multi-drug optimization experimental assessment for a first attempt in the first iteration. The adjusted R² is 0.746, which means 74.6% of the experiment data can be explained by a linear regression equation.

FIG. 11: Multi-drug optimization experimental assessment for a second attempt of the first iteration. The adjusted R² is 0.855, which means 85.5% of the experiment data can be explained by the linear regression equation.

FIG. 12: Multi-drug optimization experimental assessment for the second iteration. The adjusted R² is 0.734, which means 73.4% of the experiment data can be explained by the linear regression equation.

FIG. 13: Multi-drug optimization experimental assessment for the third iteration. The adjusted R² is 0.766, which means 76.6% of the experiment data can be explained by the linear regression equation.

FIG. 14: Multi-drug optimization experimental assessment of prescribed combinations and output. Using the linear regression equation (in FIG. 13), all five concentration levels (described in FIG. 8) are added into the respective X terms. 100% means IC₃₅ (inhibitory concentration) of that drug while 75% means a concentration of 0.75*IC₃₅. The calculated y from the equation (FIG. 13) is the output as shown here.

FIG. 15: Examples of experimentally-derived/empirically-backed response surface maps for the third iteration with 5 drugs (Bortezomib, Carfilzomib, MechloroethamineHCl, Panobinostat, and Dexamethasone). For the third iteration, there are a total of 10 of these two-drug interaction plots. In all three plots here, the surface curves vertically upwards when the concentrations of both drugs increase. Hence, these drug pairs (Bortezomib with Panobinostat, Bortezomib with Dexamethasone, and Panobinostat with Dexamethasone) have synergistic effects.

### DESCRIPTION

Multiple myeloma is a malignant monoclonal plasma cell disorder characterized by, for example, infection, anemia, abnormal calcium and creatine blood concentrations, skeletal abnormalities, and renal failure. Under current statistics, about 0.7 percent of the population will be diagnosed with myeloma at some point in their lives, and about 3.3 per 100,000 adults will die every year.

Current combination drug therapy studies in myeloma clinical trials have shown promise. Diseases, including cancer, involve numerous interactions between multiple molecular elements in a complex biological network. Multi-pronged disruption of cancer cell machinery, using drugs with complementary mechanisms, has resulted in improvements to progression-free survival and overall survival of patients. Compared to single drug therapy, rationally-designed combinatorial treatments reduce systemic toxicity, accounts for tumor heterogeneity, and overcomes drug resistance.

Besides identifying a desirable drug combination, a major challenge to combinatorial therapy is determining individual drug dosages or drug dosage ratios. Current additive design often uses the maximum tolerated dosages, determined in single drug treatment regime, for the multiple drug treatment regimes. However, such dosage levels are often higher than necessary, and recent research is advocating the use of optimal effective dosages. Reduced side effects and better drug efficacy can be achieved with such optimal effective dosages.

With an array of U.S. Food and Drug administration (FDA)-approved drugs for multiple myeloma along with FDA-approved drugs for other malignant disorders, and to allow for determining optimal effective dosages of each drug in combination therapy, a rapid and effective optimization platform is desirable. Embodiments of this disclosure are directed to systemically designed and empirically-backed optimal drug combinations using a mechanism-independent optimization platform. The results are achieved in a few months while conventional drug discovery, which produces non-optimal combinations based on additive design, can take up to one decade. Output experimental data is based on cell viabilities of healthy control cells and cancer cells. Since the goal is to maximize cancer cell death while minimizing patient toxicity, the mechanism-independent nature of the optimization platform employed in this disclosure can markedly reduce the risk of drug development and pinpoint the most effective drug combinations that are simultaneously optimized for several parameters, such as efficacy and safety.

In some embodiments, optimal drug-dosage combinations are determined on the basis of in vitro studies and analysis according to a Feedback System Control (FSC) platform. Optimal drug-dosages can be further tested in an animal model of multiple myeloma, and translating to human dosages can involve extrapolation of pharmacokinetics of drugs in animals and humans. Embodiments of this disclosure can be implemented as kits of drug combinations or as fixed dose combinations along with a pharmaceutically acceptable carrier or excipient. Administration can be orally, intravenously, or other routes.

### Drug Combinations

Some embodiments of this disclosure include various combinations of known drugs. The combinations show improved efficacy and safety for treatment of multiple myeloma, compared to conventional treatments.

In some embodiments, a drug combination for treatment of Bortezomib-resistant multiple myeloma is selected from one of the following:

Bortezomib-Resistant Multiple Myeloma

Two-Drug Optimal Combinations:
(1) Decitabine and Mitomycin C
   Optimal drug-dosage combinations determined on the basis of in vitro analysis include:
   Decitabine (about IC₁₅ to about IC₄₅) and Mitomycin C (about IC_{3.75} to about IC₄₅), such as
      Decitabine (about IC₃₀/[1.503 μM]) and Mitomycin C (about IC_{7.5}/[0.559 μM])
      Decitabine (about IC₃₀/[1.503 μM]) and Mitomycin C (about IC₁₅/[1.117 μM])
      Decitabine (about IC₃₀/[1.503 μM]) and Mitomycin C (about IC_{22.5}/[1.6755 μM])
      Decitabine (about IC₃₀/[1.503 μM]) and Mitomycin C (about IC₃₀/[2.234 μM])
(2) Bortezomib and Mechlorethamine hydrochloride
   Optimal drug-dosage combinations determined on the basis of in vitro analysis include:
   Bortezomib (about IC_{7.5} to about IC₄₅) and Mechlorethamine hydrochloride (about IC_{7.5} to about IC₄₅), such as
      Bortezomib (about IC₃₀/[0.885 μM]) and Mechlorethamine hydrochloride (about IC₃₀/[1.707 μM])
      Bortezomib (about IC₁₅/[0.442 μM]) and Mechlorethamine hydrochloride (about IC₁₅/[0.853 μM])
(3) Decitabine and Mechlorethamine hydrochloride
   Optimal drug-dosage combinations determined on the basis of in vitro analysis include:
   Decitabine (about IC_{3.75} to about IC₄₅) and Mechlorethamine hydrochloride (about IC₁₅ to about IC₄₅), such as
      Decitabine (about IC₃₀/[1.707 μM]) and Mechlorethamine hydrochloride (about IC₃₀/[1.503 μM])
      Decitabine (about IC_{22.5}/[1.28 μM]) and Mechlorethamine hydrochloride (about IC₃₀/[1.503 μM])
      Decitabine (about IC₁₅/[0.853 μM]) and Mechlorethamine hydrochloride (about IC₃₀/[1.503 μM])
      Decitabine (about IC_{7.5}/[0.427 μM]) and Mechlorethamine hydrochloride (about IC₃₀/[1.503 μM])

Three-Drug Optimal Combinations:
(4) Decitabine, Mechlorethamine hydrochloride, and Mitomycin C
   Optimal drug-dosage combinations determined on the basis of in vitro analysis include:
   Decitabine (about IC₁₅ to about IC₄₅), Mechlorethamine hydrochloride (about IC₁₅ to about IC₄₅), and Mitomycin C (about IC_{3.75} to about IC₄₅), such as
      Decitabine (about IC₃₀/[1.503 μM]), Mechlorethamine hydrochloride (about IC₃₀/[1.707 μM]), and Mitomycin C (about IC_{7.5}/[0.559 μM])
      Decitabine (about IC₃₀/[1.503 μM]), Mechlorethamine hydrochloride (about IC₃₀/[1.707 μM]), and Mitomycin C (about IC₃₀/[2.234 μM])
      Decitabine (about IC₃₀/[1.503 μM]), Mechlorethamine hydrochloride (about IC_{22.5}/[1.28 μM]]), Mitomycin C (about IC₃₀/[2.234 μM])
(5) Bortezomib, Decitabine, and Mitomycin C
   Optimal drug-dosage combinations determined on the basis of in vitro analysis include:
   Bortezomib (about IC₁₅ to about IC₄₅), Decitabine (about IC₁₅ to about IC₄₅), and Mitomycin C (about IC₁₅ to about IC₄₅), such as
      Bortezomib (about IC₃₀/[0.885 μM]), Decitabine (about IC₃₀/[1.503 μM]), and Mitomycin C (about IC₃₀/[2.234 μM])
(6) Bortezomib, Mechlorethamine hydrochloride, and Decitabine
   Optimal drug-dosage combinations determined on the basis of in vitro analysis include:
   Bortezomib (about IC₃₀/[0.885 μM]), Mechlorethamine hydrochloride (about IC₃₀/[1.707 μM]), and Decitabine (about IC₃₀/[1.503 μM]), such as
      Bortezomib (about IC₃₀/[0.885 μM]), Mechlorethamine hydrochloride (about IC₃₀/[1.707 μM]), and Decitabine (about IC₃₀/[1.503 μM])
      Bortezomib (about IC₃₀/[0.885 μM]), Mechlorethamine hydrochloride (about IC_{22.5}/[1.28 μM]), and Decitabine (about IC₃₀/[1.503 μM])
      Bortezomib (about IC_{22.5}/[0.664 μM]), Mechlorethamine hydrochloride (about IC₃₀/[1.707 μM]), and Decitabine (about IC₃₀/[1.503 μM])
      Bortezomib (about IC₁₅/[0.442 μM]), Mechlorethamine hydrochloride (about IC₃₀/[1.707 μM]), and Decitabine (about IC₃₀/[1.503 μM])

Four-Drug Optimal Combinations:
(7) Bortezomib, Mechlorethamine hydrochloride, Decitabine, and Mitomycin C
   Optimal drug-dosage combinations determined on the basis of in vitro analysis include:
   Bortezomib (about IC₁₅ to about IC₄₅), Mechlorethamine hydrochloride (about IC_{3.75} to about IC₄₅), Decitabine (about IC_{3.75} to about IC₄₅), and Mitomycin C (about IC₁₅ to about IC₄₅), such as
      Bortezomib (about IC₃₀/[0.885 μM]), Mechlorethamine hydrochloride (about IC_{7.5}/[0.427 μM]), Decitabine (about IC_{7.5}/[0.376 μM]), and Mitomycin C (about IC₃₀/[2.234])
      Bortezomib (about IC₃₀/[0.885 μM]), Mechlorethamine hydrochloride (about IC_{7.5}/[0.427 μM]), Decitabine (about IC_{7.5}/[0.376 μM]), and Mitomycin C (about IC₃₀/[2.234 μM])
      Bortezomib (about IC₃₀/[0.885), Mechlorethamine hydrochloride (about IC₁₅/[0.853 μM]), Decitabine (about IC_{7.5}/[0.376 μM]), and Mitomycin C (about IC₃₀/[2.234 μM])
      Bortezomib (about IC₃₀/[0.885 μM]), Mechlorethamine hydrochloride (about IC_{22.5}/[1.28 μM]), Decitabine (about IC_{7.5}/[0.376 μM]), and Mitomycin C (about IC₃₀/[2.234 μM])
      Bortezomib (about IC₃₀/[0.885 μM]), Mechlorethamine hydrochloride (about IC₃₀/[1.7067 μM]), Decitabine (about IC_{7.5}/[0.376 μM]), and Mitomycin C (about IC₃₀/[2.234 μM])
      Bortezomib (about IC₃₀/[0.885 μM]), Mechlorethamine hydrochloride (about IC_{7.5}/[0.427 μM]), Decitabine (about IC₁₅/[0.7515 μM]), and Mitomycin C (about IC₃₀/[2.234 μM])
      Bortezomib (about IC₃₀/[0.885 μM]) Mechlorethamine hydrochloride (about IC_{22.5}/[1.28 μM]), Decitabine (about IC₁₅/[0.7515 μM]), and Mitomycin C (about IC₃₀/[2.234 μM])
      Bortezomib (about IC₃₀/[0.885 μM]) Mechlorethamine hydrochloride (about IC_{22.5}/[1.28 μM]), Decitabine (about IC₁₅/[0.7515 μM]), and Mitomycin C (about IC₃₀/[2.234 μM])
      Bortezomib (about IC₃₀/[0.885 μM]) Mechlorethamine hydrochloride (about IC₃₀/[1.7067 μM]), Decitabine (about IC₁₅/[0.7515 μM]), and Mitomycin C (about IC₃₀/[2.234 μM])
      Bortezomib (about IC₃₀/[0.885 μM]) Mechlorethamine hydrochloride (about IC_{7.5}/[0.427 μM]), Decitabine (about IC₃₀/[1.127 μM]), and Mitomycin C (about IC₃₀/[2.234 μM])
      Bortezomib (about IC₃₀/[0.885 μM]) Mechlorethamine hydrochloride (about IC_{22.5}/[1.28 μM]), Decitabine (about IC₃₀/[1.127 μM]), and Mitomycin C (about IC₃₀/[2.234 μM])

In some embodiments, a drug combination for treatment of Bortezomib-sensitive multiple myeloma is selected from one of the following:

Bortezomib-Sensitive Multiple Myeloma

Two-Drug Optimal Combinations:
(8) Bortezomib and Dexamethasone
   Optimal drug-dosage combinations determined on the basis of in vitro analysis include:
   Bortezomib (about IC₃₅/[2.375E-3 μM]) and Dexamethasone (about

IC₃₅/[2.689E-3 μM])

Three-Drug Optimal Combinations:
(9) Bortezomib, Panobinostat, and Dexamethasone
   Optimal drug-dosage combinations determined on the basis of in vitro analysis include:
   Bortezomib (about IC₃₅), Panobinostat (about IC_{8.75} to about IC₃₅), and Dexamethasone (about IC₃₅), such as
      Bortezomib (about IC₃₅/[2.375E-3 μM]), Panobinostat (about IC₃₅/[1.195E-3 μM]), and Dexamethasone (about IC₃₅/[2.689E-3 μM])
      Bortezomib (about IC₃₅/[2.375E-3 μM]), Panobinostat (about IC_{26.25}/[8.734E-4 μM]), and Dexamethasone (about IC₃₅/[2.689E-3 μM])
      Bortezomib (about IC₃₅/[2.375E-3 μM]), Panobinostat (about IC_{17.5}/[5.905E-4 μM]), and Dexamethasone (about IC₃₅/[2.689E-3 μM])
      Bortezomib (about IC₃₅/[2.375E-3 μM]), Panobinostat (about IC_{8.75}/[3.239E-4 μM]), and Dexamethasone (about IC₃₅/[2.689E-3 μM])
(10) Bortezomib, Mechloroethamine hydrochloride, and Dexamethasone
   Optimal drug-dosage combinations determined on the basis of in vitro analysis include:
   Bortezomib (about IC₃₅), Mechloroethamine hydrochloride (about IC_{8.75} to about IC₃₅), and Dexamethasone (about IC₃₅), such as
      Bortezomib (about IC₃₅/[2.375E-3 μM]), Mechloroethamine hydrochloride (about IC₃₅/[6.664E-1 μM]), and Dexamethasone (about IC₃₅/[2.689E-3 μM])
      Bortezomib (about IC₃₅/[2.375E-3 μM]), Mechloroethamine hydrochloride (about IC_{26.25}/[4.597E-1 μM]), and Dexamethasone (about IC₃₅/[2.689E-3 μM])
      Bortezomib (about IC₃₅/[2.375E-3 μM]), Mechloroethamine hydrochloride (about IC_{17.5}/[1.75E-1 μM]), and Dexamethasone (about IC₃₅/[2.689E-3 μM])
      Bortezomib (about IC₃₅/[2.375E-3 μM]), Mechloroethamine hydrochloride (about IC_{8.75}/[1.418E-1 μM]), and Dexamethasone (about IC₃₅/[2.689E-3 μM])

Four-Drug Optimal Combinations:
(11) Bortezomib, Mechloroethamine hydrochloride, Panobinostat, and Dexamethasone
   Optimal drug-dosage combinations determined on the basis of in vitro analysis include:
   Bortezomib (about IC₃₅), Mechloroethamine hydrochloride (about IC_{8.75} to about IC₃₅), Panobinostat (about IC_{17.5} to about IC₃₅), and Dexamethasone (about IC₃₅), such as
      Bortezomib (about IC₃₅/[2.375E-3 μM]), Mechloroethamine hydrochloride (about IC₃₅/[6.664E-1 μM]), Panobinostat (about IC₃₅/[1.195E-3 μM]), and Dexamethasone (about IC₃₅/[2.689E-3 μM])
      Bortezomib (about IC₃₅/[2.375E-3 μM]), Mechloroethamine hydrochloride (about IC_{26.25}/[4.597E-1 μM]), Panobinostat (about IC₃₅/[1.195E-3 μM]), and Dexamethasone (about IC₃₅/[2.689E-3 μM])
      Bortezomib (about IC₃₅/[2.375E-3 μM]), Mechloroethamine hydrochloride (about IC_{17.5}/[1.75E-1 μM]), Panobinostat (about IC₃₅/[1.195E-3 μM]), and Dexamethasone (about IC₃₅/[2.689E-3 μM])
      Bortezomib (about IC₃₅/[2.375E-3 μM]), Mechloroethamine hydrochloride (about IC₃₅/[6.664E-1 μM]), Panobinostat (about IC_{26.25}/[8.734E-4 μM]), and Dexamethasone (about IC₃₅/[2.689E-3 μM])
      Bortezomib (about IC₃₅/[2.375E-3 μM]), Mechloroethamine hydrochloride (about IC_{8.75}/[1.418E-1 μM]), Panobinostat (about IC₃₅/[1.195E-3 μM]), and Dexamethasone (about IC₃₅/[2.689E-3 μM])
      Bortezomib (about IC₃₅/[2.375E-3 μM]), Mechloroethamine hydrochloride (about IC_{26.25}/[4.597E-1 μM]), Panobinostat (about IC_{26.25}/[8.734E-4 μM]), and Dexamethasone (about IC₃₅/[2.689E-3 μM])
      Bortezomib (about IC₃₅/[2.375E-3 μM]), Mechloroethamine hydrochloride (about IC₃₅/[6.664E-1 μM]), Panobinostat (about IC_{17.5}/[5.905E-4 μM]), and Dexamethasone (about IC₃₅/[2.689E-3 μM])
      Bortezomib (about IC₃₅/[2.375E-3 μM]), Mechloroethamine hydrochloride (about IC_{17.5}/[1.75E-1 μM]), Panobinostat (about IC_{26.25}/[8.734E-4 μM]), and Dexamethasone (about IC₃₅/[2.689E-3 μM])

### Methods of Treatment

Some embodiments of this disclosure include methods of treating multiple myeloma in a patient or other subject in need thereof, comprising administering to the patient a pharmaceutically effective amount of a drug combination described herein. For example, in some embodiments, the combination comprises, or alternatively consists essentially of, or yet further consists of: Decitabine and Mitomycin C. In some embodiments, the combination comprises, or alternatively consists essentially of, or yet further consists of one of the other combinations disclosed herein, such as selected from combinations (1) through (11) disclosed herein. In some embodiments, the methods of treating multiple myeloma in a patient in need thereof, do not comprise administering Bortezomib to the patient. In other embodiments, the methods of treating multiple myeloma in a patient in need thereof, comprise administering Bortezomib to the patient.

Drugs in a drug combination used in the methods of some embodiments are administered sequentially or concurrently. In some embodiments, one or two or three or four of compounds of a selected combination are delivered sequentially. In some embodiments, one or two or three or four of the compounds of the selected combination are delivered concurrently.

An administration schedule of the methods of some embodiments may be in a manner that provides a desirable therapeutic effect. For example, in some embodiments, a combination is administered once a day, twice a day or three times a day. In some embodiments, administration is continued for 2 or 4 or 6 or 8 weeks or more, or one, two, three, four or five months or more, or any value therein between. In some embodiments, a treatment regimen specifies less than 6 months of treatment, or less than 9, 12, 15, 18, 21 or 24 months.

In some embodiments, a subject in need thereof is a mammal. The mammal can be any mammal, including, for example, farm animals, such as sheep, pigs, cows, and horses; pet animals, such as dogs and cats; laboratory animals, such as rats, mice and rabbits. In some embodiments, the mammal is a human.

In some embodiments, the multiple myeloma treated is Bortezomib-resistant multiple myeloma. In some embodiments, the multiple myeloma treated is Bortezomib-sensitive multiple myeloma.

### Pharmaceutical Formulations

Some embodiments of this disclosure can be implemented as kits of drug combinations or as fixed dose combinations (FDCs) along with a pharmaceutically acceptable carrier or excipient. For example, drugs in optimal two-drug, three-drug or four-drug combinations can be combined into a single solid dose formulation for treating multiple myeloma, where dosages of the drugs in the combinations are in a proper ratio to each other.

For oral administration, liquid or solid dose formulations may be used. Some examples of oral dose formulations include tablets, gelatin capsules, pills, troches, elixirs, suspensions, syrups, wafers, chewing gum and the like. The compounds of some embodiments can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

For systemic, intracerebroventricular, intrathecal, topical, intravenous, intranasal, subcutaneous, intramuscular, or transdermal administration, formulations of the compounds useful in the methods of some embodiments may utilize conventional diluents, carriers, or excipients, which can be employed to deliver the compounds. For example, the formulations may comprise one or more of the following: a stabilizer, a surfactant (such as a nonionic, ionic, anionic, or zwitterionic surfactant), and optionally a salt and/or a buffering agent. The compounds may be delivered in the form of a solution, suspension, or in a reconstituted lyophilized form.

In some embodiments, a stabilizer may be, for example, an amino acid, such as glycine; or an oligosaccharide, such as sucrose, trehalose, lactose or a dextran. Alternatively, the stabilizer may be a sugar alcohol, such as mannitol; or a combination thereof. Other stabilizers may include Beeswax, butylated hydroxytoluene, citric acid, ethyl vanillin, gelatin, glycerin, iron oxide, lecithin, p-methoxy acetophenone, parabens, plant oils, and propylene glycol. In some embodiments, the stabilizer or combination of stabilizers constitutes from about 0.1% to about 10% by weight/weight of a formulation.

In some embodiments, a surfactant is a nonionic surfactant, such as a polysorbate. Some examples of suitable surfactants include polysorbates (e.g., Tween20, Tween80); a polyethylene glycol or a polyoxyethylene polyoxypropylene glycol, such as Pluronic F-68 at from about 0.001% by weight/volume (w/v) to about 10% (w/v).

A salt or buffering agent may be any suitable salt or buffering agent, such as sodium chloride, or sodium/potassium phosphate, respectively. In some embodiments, the buffering agent maintains the pH of the pharmaceutical composition in the range of about 5.5 to about 7.5. The salt and/or buffering agent is also useful to maintain the osmolality at a level suitable for administration to a human or other animal. In some embodiments, the salt or buffering agent is present at a roughly isotonic concentration of about 150 mM to about 300 mM.

The formulations of the compounds useful in the methods of this disclosure may additionally comprise one or more conventional additives. Some examples of such additives include a solubilizer such as glycerol or hydroxypropyl-cyclodextrin; an antioxidant such as benzalkonium chloride (a mixture of quaternary ammonium compounds, referred to as “quats”), benzyl alcohol, chloretone or chlorobutanol; anaesthetic agent such as a morphine derivative; or an isotonic agent. As a further precaution against oxidation or other spoilage, the pharmaceutical compositions may be stored under nitrogen gas in vials sealed with impermeable stoppers.

In some embodiments, the formulations of the compounds useful in the methods of this disclosure are contained in a single vehicle (e.g., a single oral dose form). For example, the pharmaceutical composition comprising a pharmaceutically effective amount of a combination of the compounds useful in the methods of this disclosure (e.g., Decitabine and Mitomycin C, or any other combination disclosed herein) may be provided in a single oral dose formulation (e.g., a single tablet, gelatin capsule, pill, troche, elixir, suspension, and so forth).

### Feedback System Control (FSC) Optimization Platform

Stimulations can be applied to direct a complex system towards a desired state, such as applying drugs to treat a patient. The types and the amplitudes (e.g., dosages) of applying these stimulations are part of input parameters that can affect the efficiency in bringing the system towards the desired state. However, N types of different drugs with M different dosages for each drug will result in M^{N} possible drug-dosage combinations. To identify an optimized or even near optimized combination by multiple tests on all possible combinations is prohibitive in practice. For example, it is not practical to perform all the possible drug-dosage combinations in in vitro or in vivo tests for finding an effective drug-dosage combination as the number of drugs and dosages increase.

Embodiments of this disclosure apply a technique that allows a rapid search for optimized combinations of input parameters to guide multi-dimensional (or multivariate) systems with multiple input parameters toward their desired states. The technique is comprised of a multi-dimensional complex system whose state is affected by input parameters along respective dimensions of a multi-dimensional parameter space. In some embodiments, the technique can efficiently operate on a large pool of input parameters (e.g., a drug pool), where the input parameters can involve complex interactions both among the parameters and with the complex system. A search technique can be used to identify at least a subset, or all, optimized combinations or sub-combinations of input parameters that produce desired states of the complex system. Taking the case of combinational drugs, for example, a large number of drugs can be evaluated to rapidly identify optimized combinations, ratios, and dosages of drugs. A parameter space sampling technique (e.g., an experimental design methodology) can guide the selection of a minimal or reduced number of tests to expose salient features of the complex system being evaluated, and to reveal a combination or sub-combination of input parameters of greater significance or impact in affecting a state of the complex system.

In some embodiments, an output (or a cost function) y is specified for a complex biological system being evaluated. Taking the case of combinational drugs tested on cell cultures, for example, the output can be a function of X, which is a vector of input parameters in an input parameter space (e.g., a combination of dosages of drugs sampled according to an experimental design methodology), and can be specified as a therapeutic window based on a viability of healthy control cells subjected to X and a viability of diseased (e.g., cancerous or tumor) cells subjected to X, where the former corresponds to safety of X, and the latter corresponds to efficacy of X. Other outputs can be defined, such as including an interaction effect among drugs of X, to account for whether the drugs interact synergistically, antagonistically, or when the effect of the drugs is additive. The output y represents an overall therapeutic outcome or response to be optimized (e.g., enhanced or maximized), and includes a combination (e.g., a weighted sum) of phenotypic contributions or responses, including safety or toxicity when X is applied to healthy control cells, and efficacy when X is applied to diseased cells. The output y can be represented as a response surface that is a function of input parameters within a multi-dimensional input parameter space. Other relevant phenotypic contributions can be included in the output y by applying proper transformations to adjust a range and scale of the phenotypic contributions, such as those related to improved tolerance, enlarged therapeutic window, reduced drug dosages, and broad reduction of side effects. Certain phenotypic responses are desirable, such as drug efficacy or drug safety, while other phenotypic responses are undesirable, such as drug toxicity or drug side effects. In the case of the latter phenotypic responses, their weighting factors serve as penalty factors in the optimization of combinatorial drugs.

In some embodiments, a response of a complex system to multiple input parameters can be represented by a low order function, such as a second order (or quadratic) equation, although a first order (or linear) equation as well as a third order (or cubic) equation are also contemplated as possible low order equations. Also, higher order functions are contemplated for other embodiments. Taking the case of combinational drugs, for example, an overall therapeutic response (as represented by the output y) can be specified as a function of drug dosages as follows:

$$y = {β}_{0} + {\sum }_{i} {β}_{i} {X}_{i} + {\sum }_{{ii}^{′}} {β}_{{ii}^{′}} {X}_{i} {X}_{{i}^{′}} + higher order terms$$

where Xᵢ is a dosage of an i^{th} drug from the pool of N drugs being evaluated, β₀ is a coefficient (e.g., a constant) representing a baseline response, βᵢ is a coefficient (e.g., a constant) representing a single drug response contribution, β_{ii′} is a coefficient (e.g., a constant) representing a drug-drug interaction contribution, and the summations run through N. If cubic and other higher order terms are omitted, then the output y can be represented by a quadratic equation of the drug dosages Xᵢ. As noted above, other representations, including third and higher order functions or the use of linear regression, are also contemplated.

For the case of N=1 (a pool of 1 drug), then:

y=β₀+β₁X₁+β₁₁X₁X₁

with a total of three constants, β₀, β₁, and β₁₁.

For the case of N=2 (a pool of 2 drugs), then:

y=β₀+β₁X₁+β₂X₂+β₁₂X₁X₂+β₁₁X₁X₁+β₂₂X₂X₂₂

with a total of six constants, β₀, β₁, β₂, β₁₂, β₁₁, and β₂₂.

More generally for N total drugs, a total number of parameters m is 1+2N+(N(N−1))/2. If one drug dosage is kept constant in the evaluation, the number of parameters m can be further reduced to 1+2(N−1)+((N−1)(N−2))/2, for N>1. Table 1 below sets forth a total number of coefficients in a quadratic cost function with respect to a total number drugs in a pool of drugs being evaluated.

TABLE 1

| | | Constants (m) |
|---|---|---|
| | | (if one drug |
| | | dosage is kept |
| Drugs (N) | Constants (m) | constant) |
| 1 | 3 | — |
| 2 | 6 | 3 |
| 3 | 10 | 6 |
| 4 | 15 | 10 |
| 5 | 21 | 15 |
| 6 | 28 | 21 |

An experimental design methodology is used to guide the selection of tests to sample an input parameter space. The experimental design methodology can allow exposure of salient features of a complex system being evaluated, and can reveal a combination or sub-combination of input parameters of greater significance or impact in affecting a state of the complex system. Selection of the experimental design methodology can be according to a particular cost function of the complex system being evaluated. Examples of experimental design methodologies include Latin hypercube sampling, central composite design, d-optimal design, orthogonal array design, full factorial design, and fractional factorial design, among others. In the case of combinational drugs, for example, an experimental design methodology can be used to guide the selection of drug dosages for in vitro tests. In connection with the experimental design methodology, possible dosages can be narrowed down into a few discrete levels.

Once tests are designed, therapeutic outcomes (e.g., phenotypic responses) are measured by testing each combination of input parameters sampled according to the experimental design methodology, such as by applying each combination of drug dosages in vitro.

Next, a representation of the cost function is fitted using values of the cost function measured or derived from the test results. Fitting of the cost function can be carried out by linear regression, Gaussian process regression, support vector machine regression, Bayesian regression, or another suitable technique. Based on the fitting performance between the test results and the fitted representation of the cost function, additional tests can be conducted to improve the accuracy of the fitted representation. Once the fitted representation with a desired accuracy is achieved, a globally or locally optimized combination of input parameters is determined or predicted using the fitted representation, such as by locating extrema using a stochastic or a deterministic optimization technique. Examples of stochastic techniques include simulated annealing, Markov chain Monte Carlo (MCMC), genetic optimization, differential evolution, and Gur game, among others. Examples of deterministic techniques include steepest descent and conjugate gradient, among others. An optimized combination of input parameters predicted from a fitted representation can be experimentally verified, such as by applying the optimized combination in vitro, in vivo, or in clinical/human tests.

Once a fitted representation with a desired accuracy is achieved for some embodiments, the significance of each input parameter and its synergistic effect with other input parameters can be identified. Non-significant input parameters that have little or no impact in affecting a state of a complex system can be dropped or omitted from an initial pool of input parameters, thereby effectively converting an initial multi-dimensional system to a refined system with a lower dimensionality. Taking the case of combinatorial drugs, for example, non-significant drugs can be identified as having low or negative values of the constants βᵢ, βᵢᵢ, and βᵢⱼ, and can be dropped from an initial pool of drugs for subsequent evaluation.

### Terminology

As used herein, the singular terms “a,” “an,” and “the” include plural referents unless the context clearly dictates otherwise. Thus, for example, reference to an object can include multiple objects unless the context clearly dictates otherwise.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

In some embodiments, Decitabine corresponds to 5-aza-2′-deoxycytidine, and is represented by the following structure, or a pharmaceutically acceptable salt thereof:

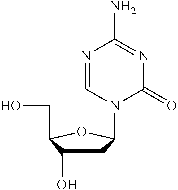

In some embodiments, Mitomycin C corresponds to a methylazirinopyrroloindoledione isolated from the bacterium Streptomyces caespitosus and other Streptomyces bacterial species, and is represented by the following structure, or a pharmaceutically acceptable salt thereof:

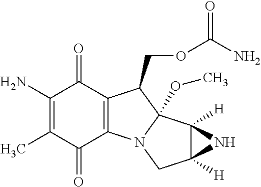 [SMILES: CO[C@@]12[C@H](COC(N)=O)C3=C(C(=O)C(C)=C(N)C3=O)N1C[C@@H]1N[C@@H]12]

In some embodiments, Bortezomib corresponds to [(1R)-3-methyl-1-({(2S)-3-phenyl-2-[(pyrazin-2-ylcarbonyl)amino]propanoyl}amino)butyl]boronic acid, and is represented by the following structure, or a pharmaceutically acceptable salt thereof:

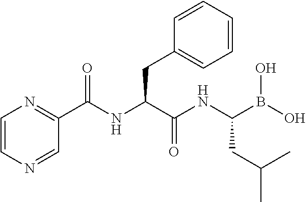 [SMILES: CC(C)C[C@H](NC(=O)[C@H](Cc1ccccc1)NC(=O)c1cnccn1)B(O)O]

In some embodiments, Mechlorethamine hydrochloride is represented by the following structure, or a pharmaceutically acceptable salt thereof:

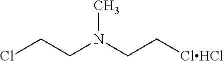

In some embodiments, Dexamethasone corresponds to (8S,9R,10S,11S,13S,14S,16R,17R)-9-Fluoro-11,17-dihydroxy-17-(2-hydroxyacetyl)-10,13,16-trimethyl-6,7,8,9,10,11,12,13,14,15,16,17-dodecahydro-3H-cyclopenta[a]phenanthren-3-one, and is represented by the following structure, or a pharmaceutically acceptable salt thereof:

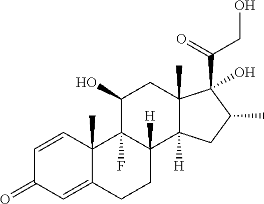

In some embodiments, Panobinostat corresponds to (2E)-N-hydroxy-3-[4-({[2-(2-methyl-1H-indol-3-yl)ethyl]amino}methyl)phenyl]acrylamide, and is represented by the following structure, or a pharmaceutically acceptable salt thereof:

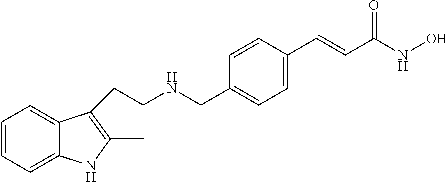 [SMILES: Cc1[nH]c2ccccc2c1CCNCc1ccc(/C=C/C(=O)NO)cc1]

In some embodiments, Carfilzomib corresponds to (2S)-N-((S)-1-((S)-4-methyl-1-((R)-2-methyloxiran-2-yl)-1-oxopentan-2-ylcarbamoyl)-2-phenylethyl)-2-((S)-2-(2-morpholino acetamido)-4-phenylbutanamido)-4-methylpentanamide, and is represented by the following structure, or a pharmaceutically acceptable salt thereof:

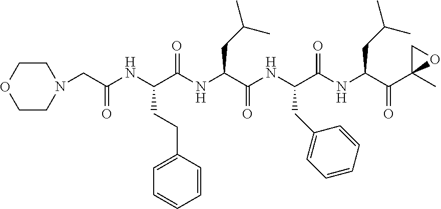 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

Those of ordinary skill in the art will appreciate that compounds of some embodiments may exhibit the phenomena of tautomerism, conformational isomerism, geometric isomerism and/or optical isomerism. Although chemical structures within the specification and claims may represent one of multiple possible tautomeric, conformational isomeric, optical isomeric and/or geometric isomeric forms, it should be understood that this disclosure encompasses any tautomeric, conformational isomeric, optical isomeric and/or geometric isomeric forms of the compounds having one or more of the utilities described herein, as well as mixtures of these various different forms.

“Tautomers” refer to isomeric forms of a compound that are in equilibrium with each other. The concentrations of the isomeric forms will depend on an environment in which the compound is found and may be different depending upon, for example, whether the compound is a solid or is in an organic or aqueous solution. For example, in aqueous solution, pyrazoles may exhibit the following isomeric forms, which are referred to as tautomers of each other:

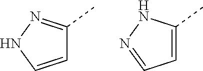

As will be understood by one of ordinary skill in the art, a wide variety of functional groups and other chemical structures may exhibit tautomerism, and all tautomers of compounds as described herein are within the scope of this disclosure.

Stereoisomers of compounds, also referred to as “optical isomers,” include all chiral, diastereomeric, and racemic forms of a chemical structure, unless the specific stereochemistry is expressly indicated. Thus, compounds used in some embodiments include enriched or resolved optical isomers at any or all asymmetric atoms as are apparent from the depictions. Both racemic and diastereomeric mixtures, as well as individual optical isomers can be isolated or synthesized so as to be substantially free of their enantiomeric or diastereomeric partners, and these are all within the scope of this disclosure.

The term “pharmaceutically acceptable” refers to a material that is not biologically or otherwise undesirable, namely the material may be incorporated into a pharmaceutical composition administered to a patient without causing undesirable biological effects or interacting in a deleterious manner with any of other components of the composition in which it is contained. When the term “pharmaceutically acceptable” is used to refer to a pharmaceutical carrier or excipient, the carrier or excipient is one that has met standards of toxicological and manufacturing testing or that is included on the Inactive Ingredient Guide prepared by the U.S. Food and Drug administration.

The term “patient” refers to any animal for which treatment is desirable. Patients may be mammals, and typically, as used herein, a patient is a human individual.

The term “pharmaceutically acceptable salt,” as used herein, represents salts or zwitterionic forms of compounds of some embodiments, which are water or oil-soluble or dispersible, which are suitable for treatment of diseases without undue toxicity, irritation, and allergic-response, which are commensurate with a reasonable benefit/risk ratio, and which are effective for their intended use. The salts can be prepared during a final isolation and purification of the compounds or separately by reacting an appropriate compound in the form of a free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of some embodiments can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, this disclosure contemplates sodium, potassium, magnesium, and calcium salts of the compounds of some embodiments and the like.

The term “solvate” is used in its broadest sense. For example, solvates include hydrates formed when a compound of some embodiments contains one or more bound water molecules.

### Working Example

The following example describes specific aspects of some embodiments of this disclosure to illustrate and provide a description for those of ordinary skill in the art. The example should not be construed as limiting this disclosure, as the example merely provides specific methodology useful in understanding and practicing some embodiments of this disclosure.

### Drug Combinations for Drug-Resistant and Drug-Sensitive Multiple Myeloma

Multiple myeloma is a plasma cell malignancy that arises initially from intramedullary tumor sites. It clinically manifests as anemia, renal failure, bone fractures or hypercalcemia and leads to significant morbidity. There have been a number of therapeutics approved for myeloma treatment that resulted in increased survival time and patients achieving complete remission for a limited period; however, multiple myeloma remains incurable. While the recent development of molecular targeted therapies represents a significant step forward in myeloma therapy, the determination of which drug combination is the best and what dosages to use in such combination becomes a significant challenge. Thus, there is a need to develop a better strategy to identify optimal drug combinations and patient-specific dosages for both efficacy and safety.

In order to achieve optimal lethal combinations for respective tumors, this example sets forth the use of FSC, a top-down approach that directs a biological system towards a desired phenotype. This serves as a cost- and time-effective tool to screen for optimal drug combinations in contrast to the traditional high-throughput screening, a bottom-up approach that involves detailed information about the pathways and is not able to provide the optimal dosage for each drug used in the combination. As an experimentally-driven, phenotypic optimization platform, FSC can be used to rationally and systematically converge upon globally optimal drug combinations and dosages in a multi-objective fashion. Applying this platform against multiple myeloma cells, including Bortezomib-resistant and Bortezomib-sensitive cells, a series of FSC-derived optimal therapeutic drug combinations are identified that outperform existing drug combinations. Additionally, FSC rationally derives optimal drug dosages and drug dosage ratios for these drug combinations, a previously unmet need in the field of therapeutic drug combination design that further increases efficacy and safety of improved drug combinations.

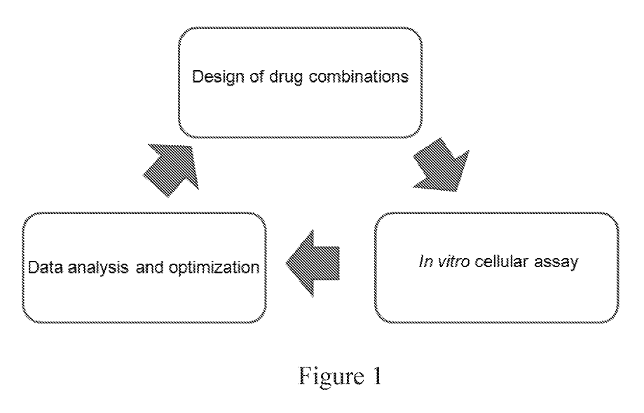

The FSC platform of this example can be represented as encompassing three stages: Design of drug combinations, in vitro cellular assay, and data analysis and optimization (see FIG. 1). Therefore, a key advantage of this approach is that the optimal drug ratios are empirically-backed and not based on prediction.

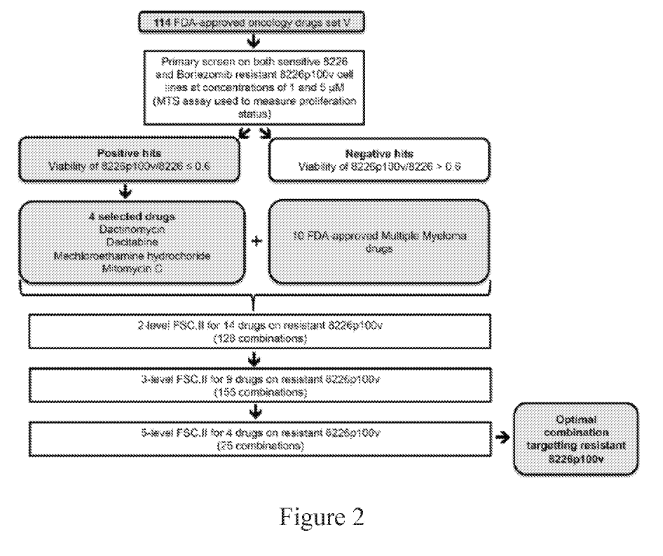

Bortezomib-resistant multiple myeloma: A general overview of a workflow for FSC is the input of drug combinations to cells, which translates into a selected output (e.g., cell viability), and this output is then analyzed. The significant drugs and drug combinations can be derived from a single analysis or refined for more improved dosage ratios through higher-level iterations (see FIG. 2). As such, this platform highlights the important drug combinations as well as their dosages that significantly affect the cells. A system of interest (in this example, Bortezomib-sensitive and Bortezomib-resistant multiple myeloma cells) were treated with drug combinations based on a design that combines a 2-level factorial design and a 3-level orthogonal array, where the levels indicate the different dosages (e.g., concentrations) of the drugs used. The initial drug screening iteration, which involves the 2-level factorial component, highlights drugs that are important from a group of drugs. After incubation of the drugs, the output or result of the experiment is then analyzed using Matlab or other computer software. The significant drugs highlighted, as indicated by p-values <0.05, are then used for the next iteration with a smaller number of drugs at 3-level and 5-level designs, indicating that three or five dosages are used. Further implementation details on the iterations are provided in the following section on Bortezomib-sensitive multiple myeloma. Using this approach, optimal drug combinations and their optimal drug dosages can be experimentally derived over 308 tested combinations compared to 36,692 combinations that would be involved to identify similar results through high-throughput screening. Further, high-throughput screening would not be able to identify and map the drug interactions that provide a fundamental explanation for the results that are observed, and further bottom-up biological experimentation would be involved to identify these drug interactions.

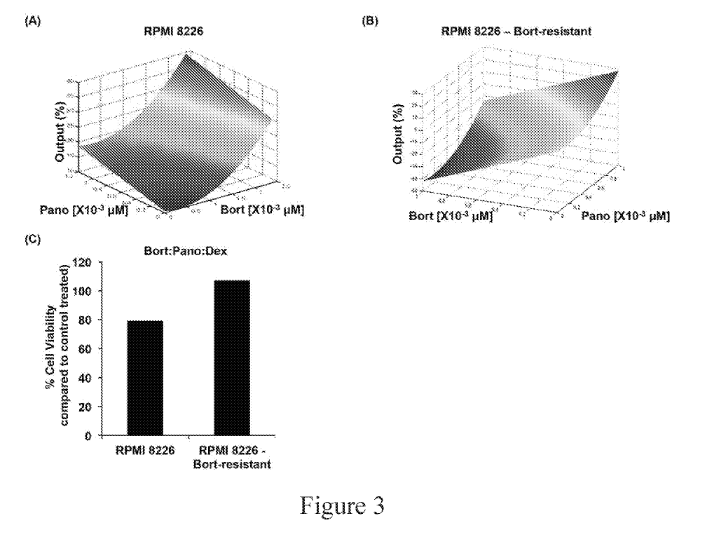

FSC was used to generate response surface maps that reliably show synergistic interactions such as Bortezomib and Panobinostat in Bortezomib-sensitive RPMI 8226 multiple myeloma cells, as well as antagonistic interactions between Bortezomib and Panobinostat in Bortezomib-resistant RPMI 8226 multiple myeloma cells. These predicted drug interactions can be confirmed by experimental data (FIG. 3). Because FSC is able to optimize for multiple parameters, drug interactions were mapped, and optimal drug combinations were derived with respect to both maximal multiple myeloma cell killing and minimal normal epithelial cell toxicity (THLE-2; immortalized normal liver epithelial cells). This is expressed as a therapeutic window (% Output) that is the difference in percentage (%) viability between treated multiple myeloma and THLE-2 cells.

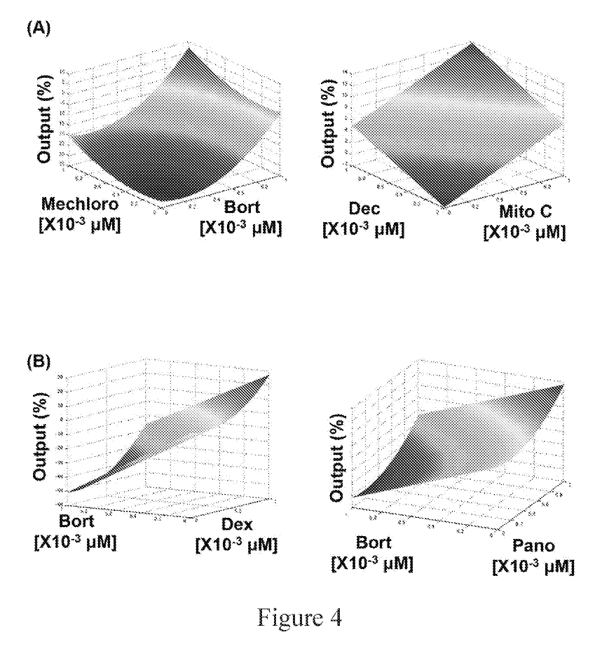

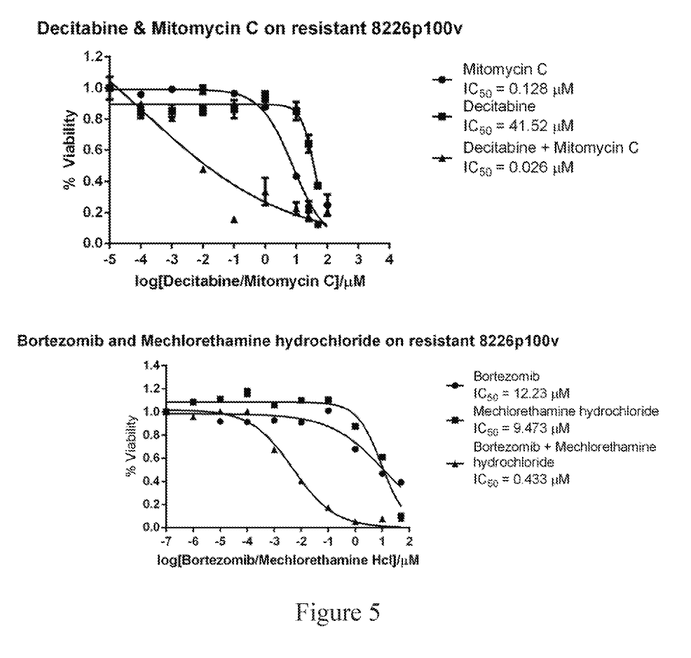

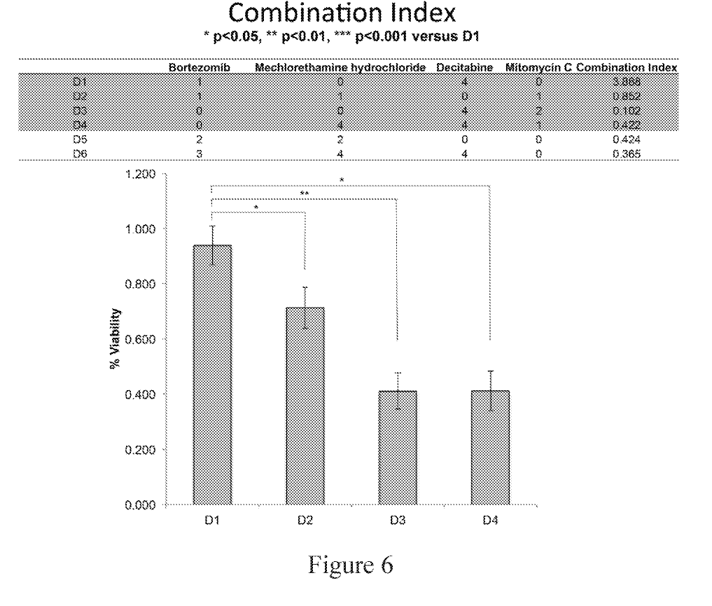

In Bortezomib-resistant multiple myeloma cells, FSC was used to identify previously undescribed synergistic and antagonist drug interactions that can inform the design of optimal drug combinations against multiple myeloma that no longer responds to Bortezomib treatment (FIG. 4). Furthermore, synergistic drug combinations were experimentally validated in dosage-response cell viability curves (FIG. 5). FIG. 4 and FIG. 5 provide experimental validation of the ability of FSC to identify previously undescribed, optimal synergistic drug combinations in a time-efficient and cost-efficient manner. Ultimately, a series of optimal 4, 3 and 2-drug combinations and optimal dosage ratios within these combinations are identified for both Bortezomib-sensitive and Bortezomib-resistant multiple myeloma. As an example, optimal 2-drug and 3-drug combinations against Bortezomib-resistant multiple myeloma are depicted with their Combination Index listed to show highly synergistic drug combinations compared to a low-ranked antagonistic drug combination (FIG. 6). A list of optimal 4, 3 and 2-drug combinations and optimal dosage ratios within these combinations for Bortezomib-resistant multiple myeloma is set forth in Table 2 below. Surprisingly, Bortezomib-containing combinations are shown to be effective in the Bortezomib-resistant cell line when combined with Mechlorethamine Hydrochloride, which appears to sensitize the cells to Bortezomib despite their resistance.

TABLE 2
A list of optimal 4, 3 and 2-drug combinations and optimal dosage ratios
within these combinations for Bortezomib-resistant multiple myeloma.

| | Bortezomib | Mechloroethamine Hcl | Decitabine | Mitomycin C |
|---|---|---|---|---|
| Optimal | 0 | 0 | IC30/[1.503 μM] | IC7.5/[0.559] |
| 2-drug combos | 0 | 0 | IC30/[1.503 μM] | IC15/[1.117] |
| (Bort-resistant) | 0 | 0 | IC30/[1.503 μM] | IC22.5/[1.6755] |
| | 0 | 0 | IC30/[1.503 μM] | IC30/[2.234] |
| | IC30/[0.885 μM] | IC30/[1.707 μM] | 0 | 0 |
| | IC15/[0.442 μM] | IC15/[0.853 μM] | 0 | 0 |
| | 0 | IC30/[1.707 μM] | IC30/[1.503 μM] | 0 |
| | 0 | IC22.5/[1.28 μM] | IC30/[1.503 μM] | 0 |
| | 0 | IC15/[0.853 μM] | IC30/[1.503 μM] | 0 |
| | 0 | IC7.5/[0.427 μM] | IC30/[1.503 μM] | 0 |
| Optimal | 0 | IC30/[1.707 μM] | IC30/[1.503 μM] | IC7.5/[0.559 μM] |
| 3-drug combos | 0 | IC30/[1.707 μM] | IC30/[1.503 μM] | IC30/[2.234 μM] |
| (Bort-resistant) | 0 | IC22.5/[1.28 μM] | IC30/[1.503 μM] | IC30/[2.234 μM] |
| | IC30/[0.885 μM] | 0 | IC30/[1.503 μM] | IC30/[2.234 μM] |
| | IC30/[0.885 μM] | IC30/[1.707 μM] | IC30/[1.503 μM] | 0 |
| | IC30/[0.885 μM] | IC22.5/[1.28 μM] | IC30/[1.503 μM] | 0 |
| | IC22.5/[0.664 μM] | IC30/[1.707 μM] | IC30/[1.503 μM] | 0 |
| | IC15/[0.442 μM] | IC30/[1.707 μM] | IC30/[1.503 μM] | 0 |
| Optimal | IC30/[0.885 μM] | IC7.5/[0.427 μM] | IC7.5/[0.376 μM] | IC30/[2.234 μM] |
| 4-drug combos | IC30/[0.885 μM] | IC15/[0.853 μM] | IC7.5/[0.376 μM] | IC30/[2.234 μM] |
| (Bort-resistant) | IC30/[0.885 μM] | IC22.5/[1.28 μM] | IC7.5/[0.376 μM] | IC30/[2.234 μM] |
| | IC30/[0.885 μM] | IC30/[1.7067 μM] | IC7.5/[0.376 μM] | IC30/[2.234 μM] |
| | IC30/[0.885 μM] | IC7.5/[0.427 μM] | IC15/[0.7515 μM] | IC30/[2.234 μM] |
| | IC30/[0.885 μM] | IC22.5/[1.28 μM] | IC15/[0.7515 μM] | IC30/[2.234 μM] |
| | IC30/[0.885 μM] | IC22.5/[1.28 μM] | IC15/[0.7515 μM] | IC30/[2.234 μM] |
| | IC30/[0.885 μM] | IC30/[1.7067 μM] | IC15/[0.7515 μM] | IC30/[2.234 μM] |
| | IC30/[0.885 μM] | IC7.5/[0.427 μM] | IC30/[1.127 μM] | IC30/[2.234 μM] |
| | IC30/[0.885 μM] | IC22.5/[1.28 μM] | IC30/[1.127 μM] | IC30/[2.234 μM] |

Bortezomib-sensitive multiple myeloma: Using a similar workflow as shown in FIG. 2, initial screening was performed on fourteen FDA-approved drugs, and two iterations (experimental runs) were performed to screen out unfavorable drug candidates. A third iteration was performed using five favorable drug candidates to determine optimal drug dosages.

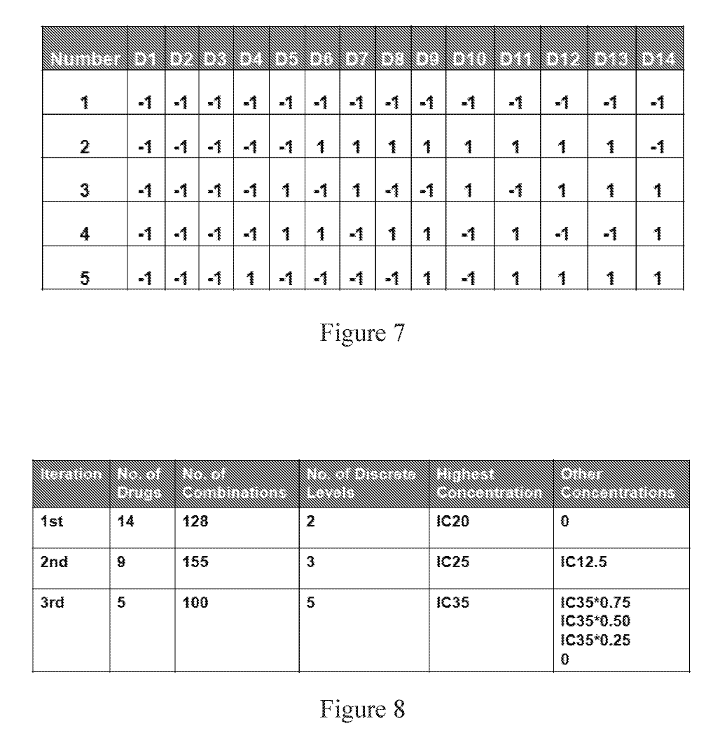

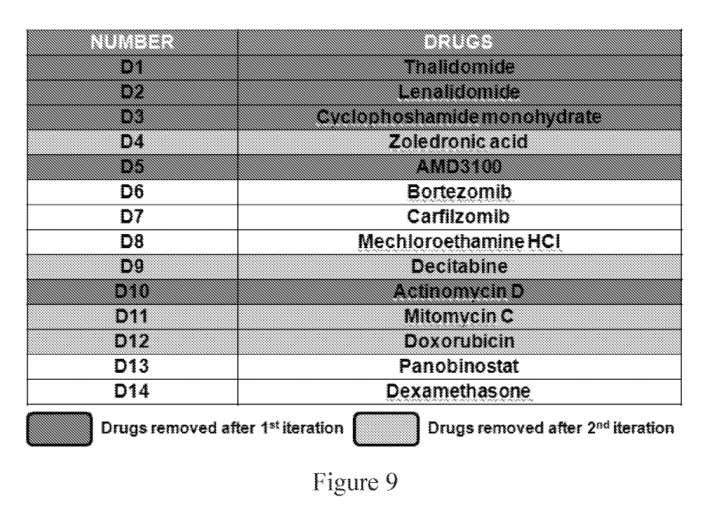

In each iteration (experiment run), a list of drug combinations used is based on an experimental design methodology that aims to reduce the number of experiments to obtain sufficient information to build a mathematical equation. An excerpt of the first iteration is shown in FIG. 7, and details of the design of the three iterations are shown in FIG. 8.

After the list of drug combinations is determined, the drug combinations are applied to an in vitro cellular assay. A healthy control cell (THLE-2) is used to determine liver toxicity caused by drug treatment. Drug efficacy is determined using the B lymphocyte cancer cell (RPMI 8226). Favorable drug combinations would maximize cancer cell killing while minimizing health cell death. Hence, the goal of the FSC implementation of this example is to maximize the output, which is the cell viability of THLE-2 minus the cell viability of RPMI 8226. The experiments are conducted in a rapid, high-throughput manner using an automated liquid handler machine.

The output of the drug combinations are run through the programming software MATLAB to provide a linear regression analysis to assist in reconciling the actual experimental results. This is a mathematical equation, with terms up to the power of 2 (or more), which summarizes the experimental observations. While each conducted experiment involves fewer than 200 drug combinations, the generated equation can prescribe the output of many more drug combinations and drug dosages.

y=β₀+β₁X₁+ . . . +βₙXₙ+β₁₂X₁X₂+ . . . +βₘₙXₘXₙ+β₁₁X₁²+ . . . +βₙₙXₙ²

As shown in the above equation, β refers to a coefficient (e.g., a constant), while X₁ refers to a dosage of drug 1, and so forth. Similarly, β₁₂ refers to a coefficient of an interaction term for X₁ and X₂ (interaction of drug 1 and drug 2), and so forth.

The programming software also provides the R² and adjusted R² values, which explain the percentage accuracy of the mathematical equation. An adjusted R² value of, for example, 0.90 means 90% of the experimental data can be accounted for by the equation. The fitting correlation term is the square root of this R² value.

Results

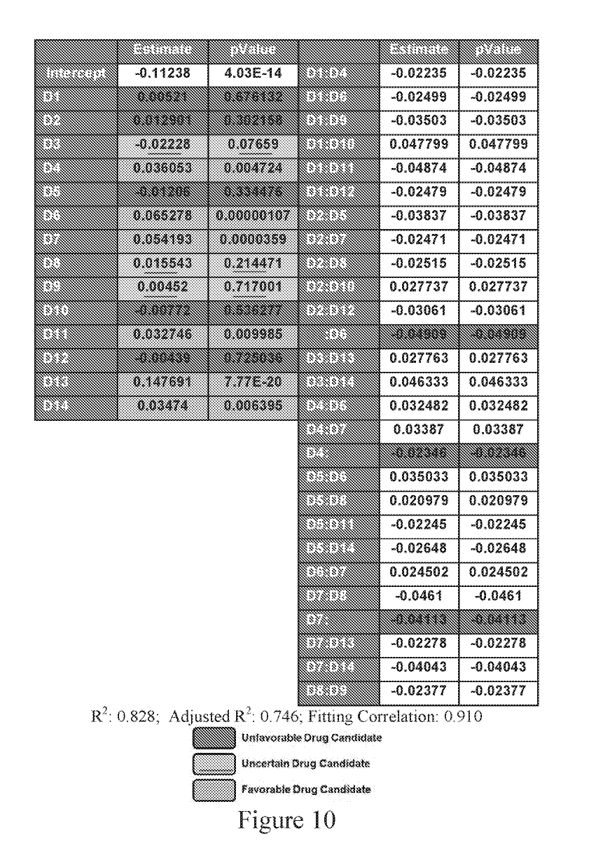

First iteration (first attempt): In the first and second iterations, the process of selecting and eliminating drug candidates involves examining the experimental coefficients at the single and two-drug level. Since the output is the viability of control cells (THLE2) minus the viability of cancer cells (RPMI 8226), the aim is to maximize the output. Observing the single-drug level, undesirable drug candidates have either, or both, negative coefficients and non-significant p values (>0.05). D1, D2, D5, D10 and D12 (Thalidomide, Lenalidomide, AMD3100, Actinomycin, and Doxorubicin) are thus eliminated. Desirable drug candidates have positive coefficients and are significant at the 5% level. These are D4, D6, D7, D11, D13 and D14 (Zoledronic Acid, Bortezomib, Carfilzomib, Mitomycin C, Panobinostat, and Dexamethasone). D3, D8, and D9 (Cyclophoshamide monohydrate, MechloroethamineHCl, and Decitabine) are inconclusive. Examining two-drug interactions, D3 and D9 can be eliminated because the coefficients of the interaction terms are negative when these drugs interact with desirable candidates (D3|D6, D4|D9, and D7|D9) (see FIG. 10).

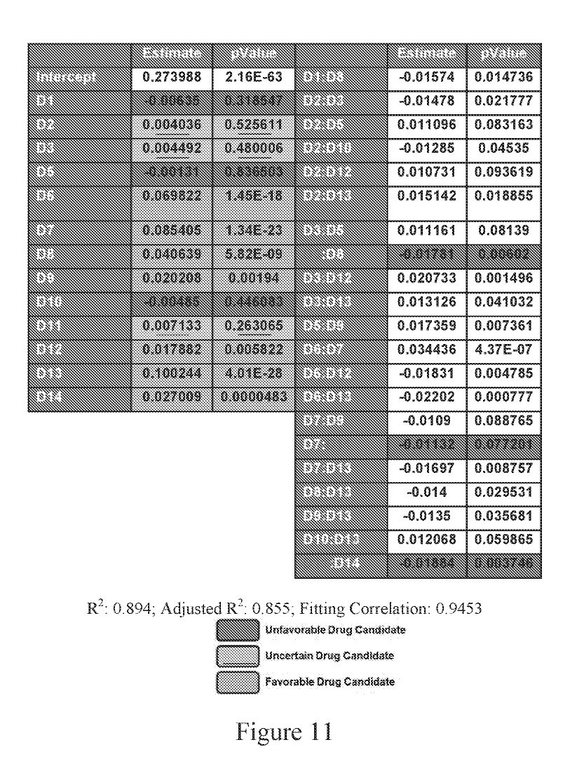

First iteration (second attempt): Based on the negative coefficients (estimates) and non-significant p values (>0.05), D1, D5, and D10 (Thalidomide, AMD3100, Actinomycin D) can be eliminated. Desirable drug candidates have positive coefficients and are significant at the 5% level. These are D6, D7, D8, D9, D12, D13 and D14 (Zoledronic Acid, Bortezomib, Carfilzomib, Mitomycin C, Panobinostat, and Dexamethasone). D2, D3, and D11 (Lenalidomide, Cyclophoshamide monohydrate, and Mitomycin C) are inconclusive. Examining two-drug interactions, D3 and D11 can be eliminated because the coefficients of the interaction terms are negative when these drugs (D3 and D11) interact with desirable candidates (D3|D8, D7|D11, and D11|D14) (see FIG. 11).

The desirable drugs from the first attempt are D4, D6, D7, D11, D13 and D14 while the desirable drugs from the second attempt are D6, D7, D8, D9, D12, D13 and D14. The common drugs in these two sets are D6, D7, D13 and D14 (Bortezomib, Carfilzomib, Panobinostat and Dexamethasone). The drugs in these two sets are combined for the second iteration: D4, D6, D7, D8, D9, D11, D12, D13 and D14 (Zoledronic acid, Bortezomib, Carfilzomib, MechloroethamineHCl, Decitabine, Mitomycin C, Doxorubicin, Panobinostat, and Dexamethasone).

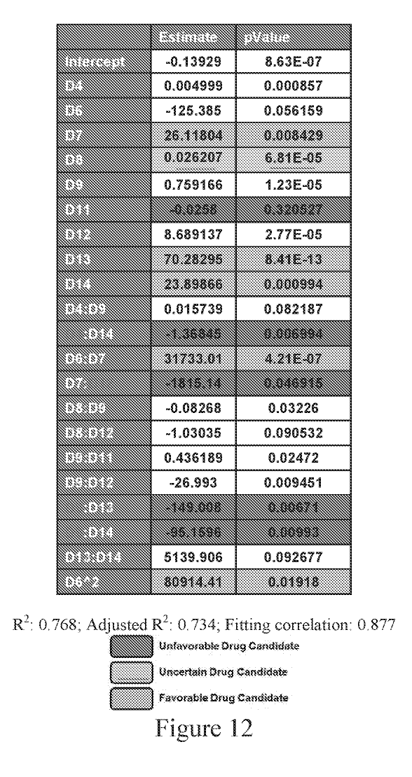

Second iteration: D7, D13, and D14 are desirable candidates (Carfilzomib, Panobinostat, and Dexamethasone). D11 (Mitomycin C) can be removed based on its negative coefficient and non-significant p value (>0.05). While D6 (Bortezomib) has a negative coefficient at the single drug level, the interaction and squared terms (D6|D7 and D6̂2) are highly positive and significant. Moreover, Bortezomib has been shown to be highly favorable in the first iteration experiments. D9 (Decitabine) can be removed because it negatively interacts with D13 and D14. D4 (Zoledronic acid) negatively interacts with D14, while D12 (Doxorubicin) negatively interacts with D7. Hence, both D4 and D12 can be eliminated. D8 (MechloroethamineHCl) presents a relatively small positive coefficient and is thus inconclusive, and is retained for the third iteration (see FIG. 12).

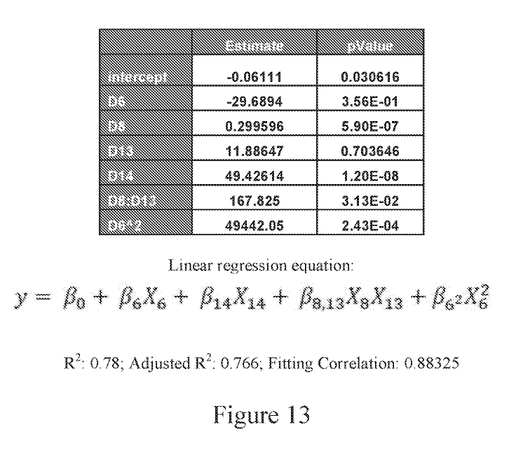

Third iteration: The fitting correlation and the adjusted R² values are high (88.3% and 76.6%), which indicates that the equation accounts for a high variability in the output. Even though D6 (Bortezomib) has a highly negative drug effect at the single drug level, the squared term (D6̂2) has a markedly positive effect. Hence, D6 is retained in the combination. D7 (Carfilzomib) is removed entirely from the equation based on non-significance. Note that the p values for D6 (Bortezomib) and D13 (Panobinostat) are not significant at the 5% significance level (p>0.05). At this iteration, however, the aim is to determine the optimal drug dosages in vitro (see FIG. 13).

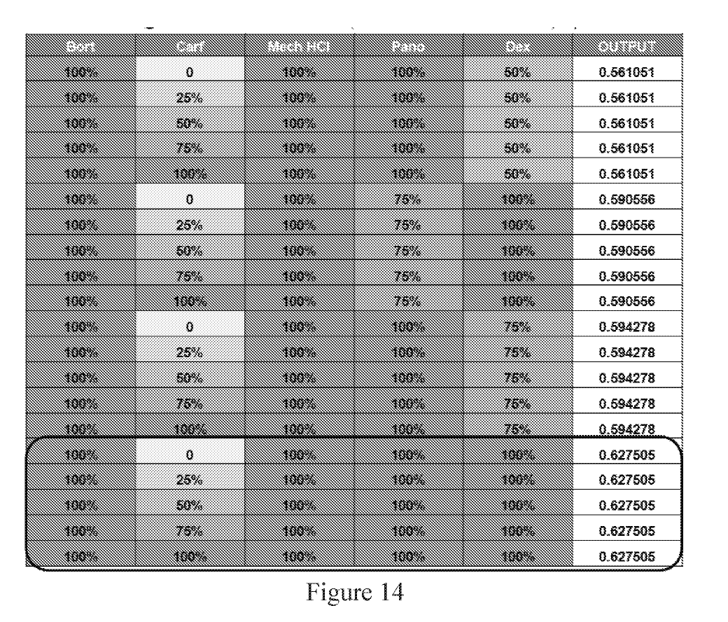

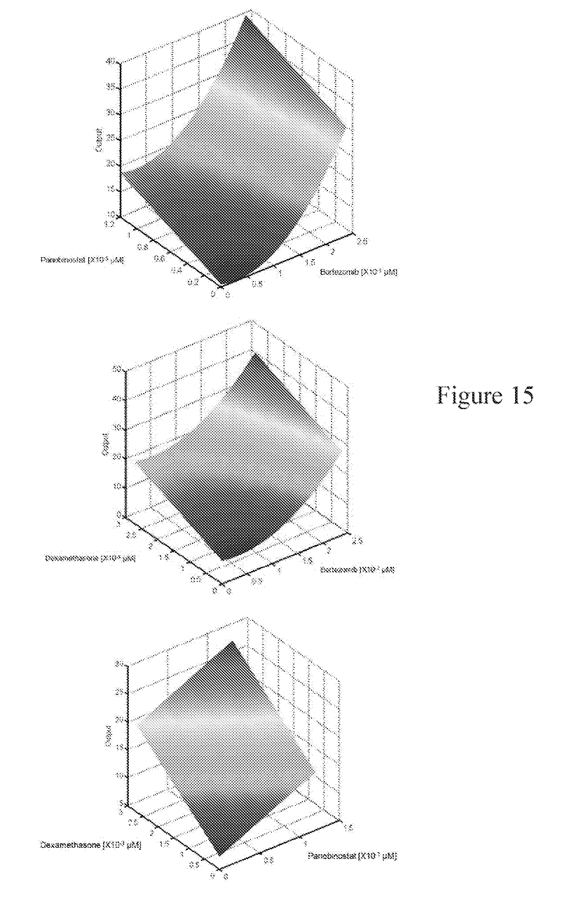

The best combinations and outputs are shown in FIG. 14. With the other four drugs (Bortezomib, MechloroethamineHCl, Panobinostat, and Dexamethasone) kept at 100%, the presence or absence of Carfilzomib does not change the output (0.6275) (refer to box). Hence, Carfilzomib may potentially be eliminated from the combination. FIG. 15 shows examples of experimentally-derived/empirically-backed response surface maps for the third iteration, and a list of optimal 5, 4, 3 and 2-drug combinations and optimal dosages within these combinations for Bortezomib-sensitive multiple myeloma is set forth in Table 3 below.

TABLE 3
A list of optimal 4, 3 and 2-drug combinations and optimal dosages within
these combinations for Bortezomib-sensitive multiple myeloma.

| | Bortezomib | Carfilzomib | MechloroethamineHCl | Panobinostal | Dexamethasone |
|---|---|---|---|---|---|
| 2 drug | IC35/[2.375E−3 μM] | 0 | 0 | IC35/[1.195E−3 μM] | IC35/[2.689E−3 μM] |
| 3 drug | IC35/[2.375E−3 μM] | 0 | 0 | IC26.25/[8.734E−4 μM] | IC35/[2.689E−3 μM] |
| | IC35/[2.375E−3 μM] | 0 | 0 | IC17.5/[5.905E−4 μM] | IC35/[2.689E−3 μM] |
| | IC35/[2.375E−3 μM] | 0 | IC35/[6.664E−1 μM] | 0 | IC35/[2.689E−3 μM] |
| | IC35/[2.375E−3 μM] | 0 | IC26.25/[4.597E−1 μM] | 0 | IC35/[2.689E−3 μM] |
| | IC35/[2.375E−3 μM] | 0 | 0 | IC8.75/[3.239E−4 μM] | IC35/[2.689E−3 μM] |
| | IC35/[2.375E−3 μM] | 0 | IC17.55/[1.75E−1 μM] | 0 | IC35/[2.689E−3 μM] |
| | IC35/[2.375E−3 μM] | 0 | IC8.75/[1.418E−1 μM] | 0 | IC35/[2.689E−3 μM] |
| 4 drug | IC35/[2.375E−3 μM] | 0 | IC35/[6.664E−1 μM] | IC35/[1.195E−3 μM] | IC35/[2.689E−3 μM] |
| | IC35/[2.375E−3 μM] | 0 | IC26.25/[4.597E−1 μM] | IC35/[1.195E−3 μM] | IC35/[2.689E−3 μM] |
| | IC35/[2.375E−3 μM] | 0 | IC17.5/[1.75E−1 μM] | IC35/[1.195E−3 μM] | IC35/[2.689E−3 μM] |
| | IC35/[2.375E−3 μM] | 0 | IC35/[6.664E−1 μM] | IC26.25/[8.734E−4 μM] | IC35/[2.689E−3 μM] |
| | IC35/[2.375E−3 μM] | 0 | IC8.75/[1.418E−1 μM] | IC35/[1.195E−3 μM] | IC35/[2.689E−3 μM] |
| | IC35/[2.375E−3 μM] | 0 | IC26.25/[4.597E−1 μM] | IC26.25/[8.734E−4 μM] | IC35/[2.689E−3 μM] |
| | IC35/[2.375E−3 μM] | 0 | IC35/[6.664E−1 μM] | IC17.5/[5.905E−4 μM] | IC35/[2.689E−3 μM] |
| | IC35/[2.375E−3 μM] | 0 | IC17.5/[1.75E−1 μM] | IC26.25/[8.734E−4 μM] | IC35/[2.689E−3 μM] |

Conclusion

Current available drug combinations are not optimized and as such FSC is used to bridge this gap by determining optimized drug combinations that effectively target the system of interest. Additionally, current approaches such as high-throughput screening are time- and cost-intensive approaches that provide less information for drug combinations identified. In this example for the Bortezomib-sensitive case, 308 FSC-derived experimental combinations were able to identify optimal drug combinations with optimal drug dosages compared to 36,692 combinations that would be involved in high-throughput screening. Additionally, synergistic and antagonistic drug interaction response surface maps can be generated to identify why optimal combinations were optimal whereas further experimentation would be involved beyond high-throughput screening to determine this information.

In this example, FSC is applied towards the development of improved therapeutic options against multiple myeloma, a serious disease with poor outcome. Optimal drug combinations and optimal drug dosages are identified for both Bortezomib-sensitive multiple myeloma cells as well as Bortezomib-resistant multiple myeloma cells. This is important as Bortezomib is a drug that is used in a second-line treatment in multiple myeloma with great efficacy; however, the drug dosage/ratios of Bortezomib in combination with other drugs like Panobinostat and Dexamethasone is currently not optimized. Additionally, eventually most or all patients will become resistant to Bortezomib and have to be subjected to a third-line treatment, and this example sets forth the identification of a number of F SC-derived optimal drug combinations that can overcome this resistance and effectively treat multiple myeloma following the development of Bortezomib resistance.

### Embodiments

The following embodiments are within the scope of this disclosure.

Embodiment 1. A pharmaceutical composition comprising a pharmaceutically effective amount of each drug in a drug combination selected from the group consisting of:
decitabine and mitomycin C;
bortezomib and mechlorethamine hydrochloride;
decitabine and mechlorethamine hydrochloride;
decitabine, mechlorethamine hydrochloride, and mitomycin C;
bortezomib, decitabine, and mitomycin C;
bortezomib, mechlorethamine hydrochloride, and decitabine; and
bortezomib, mechlorethamine hydrochloride, decitabine, and mitomycin C.

Embodiment 2. A pharmaceutical composition comprising a pharmaceutically effective amount of each drug in a drug combination selected from the group consisting of:
bortezomib and dexamethasone;
bortezomib, panobinostat, and dexamethasone;
bortezomib, mechloroethamine hydrochloride, and dexamethasone; and
bortezomib, mechloroethamine hydrochloride, panobinostat, and dexamethasone.

Embodiment 3. The pharmaceutical composition of Embodiment 1 or 2, wherein the pharmaceutically effective amount, or dosage, of each respective drug in the drug combination is below a maximum tolerated dosage of that respective drug.

Embodiment 4. A method of treating bortezomib-resistant multiple myeloma in a subject in need thereof, comprising administering to the subject a pharmaceutically effective amount of each drug in a drug combination selected from the group consisting of:
decitabine and mitomycin C;
bortezomib and mechlorethamine hydrochloride;
decitabine and mechlorethamine hydrochloride;
decitabine, mechlorethamine hydrochloride, and mitomycin C;
bortezomib, decitabine, and mitomycin C;
bortezomib, mechlorethamine hydrochloride, and decitabine; and
bortezomib, mechlorethamine hydrochloride, decitabine, and mitomycin C.

Embodiment 5. A method of treating bortezomib-resistant multiple myeloma in a subject in need thereof, comprising administering to the subject a pharmaceutically effective amount of each drug in a drug combination comprising bortezomib and at least one additional drug selected from the group consisting of mechlorethamine hydrochloride, decitabine, and mitomycin C.

Embodiment 6. A method of treating Bortezomib-sensitive multiple myeloma in a subject in need thereof, comprising administering to the subject a pharmaceutically effective amount of each drug in a drug combination selected from the group consisting of:
bortezomib and dexamethasone;
bortezomib, panobinostat, and dexamethasone;
bortezomib, mechloroethamine hydrochloride, and dexamethasone; and
bortezomib, mechloroethamine hydrochloride, panobinostat, and dexamethasone.

Embodiment 7. A method of treating multiple myeloma in a subject in need thereof, comprising administering to the subject a pharmaceutically effective amount of each drug in a drug combination comprising decitabine and at least one additional drug different from decitabine.

Embodiment 8. A method of treating multiple myeloma in a subject in need thereof, comprising administering to the subject a pharmaceutically effective amount of each drug in a drug combination comprising mechlorethamine hydrochloride and at least one additional drug different from mechlorethamine hydrochloride.

Embodiment 9. A method of treating multiple myeloma in a subject in need thereof, comprising administering to the subject a pharmaceutically effective amount of each drug in a drug combination comprising mitomycin C and at least one additional drug different from mitomycin C.

Embodiment 10. The method of any one of Embodiments 4-9, wherein two or more drugs in the drug combination are administered sequentially.

Embodiment 11. The method of any one of Embodiments 4-9, wherein two or more drugs in the drug combination are administered concurrently.

Embodiment 12. The method of any one of Embodiments 4-9, wherein the subject is a mammal.

Embodiment 13. The method of any one of Embodiments 4-9, wherein the subject is a human.

Embodiment 14. The method of any one of Embodiments 4-9, wherein the pharmaceutically effective amount, or dosage, of each respective drug in the drug combination is below a maximum tolerated dosage of that respective drug.

While this disclosure has been described with reference to the specific embodiments thereof, it should be understood by those skilled in the art that various changes may be made and equivalents may be substituted without departing from the true spirit and scope of this disclosure as defined by the appended claims. In addition, many modifications may be made to adapt a particular situation, material, composition of matter, method, operation or operations, to the objective, spirit and scope of this disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of this disclosure. Accordingly, unless specifically indicated herein, the order and grouping of the operations is not a limitation of this disclosure.

1. A pharmaceutical composition comprising a pharmaceutically effective amount of each drug in a drug combination selected from the group consisting of:
decitabine and mitomycin C;
bortezomib and mechlorethamine hydrochloride;
decitabine and mechlorethamine hydrochloride;
decitabine, mechlorethamine hydrochloride, and mitomycin C;
bortezomib, decitabine, and mitomycin C;
bortezomib, mechlorethamine hydrochloride, and decitabine; and
bortezomib, mechlorethamine hydrochloride, decitabine, and mitomycin C.

2. A pharmaceutical composition comprising a pharmaceutically effective amount of each drug in a drug combination selected from the group consisting of:
bortezomib and dexamethasone;
bortezomib, panobinostat, and dexamethasone;
bortezomib, mechloroethamine hydrochloride, and dexamethasone; and
bortezomib, mechloroethamine hydrochloride, panobinostat, and dexamethasone.

3. The pharmaceutical composition of claim 1, wherein the pharmaceutically effective amount of each drug in the drug combination is below a maximum tolerated dosage.

4. A method of treating bortezomib-resistant multiple myeloma in a subject in need thereof, comprising:
administering to the subject a pharmaceutically effective amount of each drug in a drug combination selected from the group consisting of:
decitabine and mitomycin C;
bortezomib and mechlorethamine hydrochloride;
decitabine and mechlorethamine hydrochloride;
decitabine, mechlorethamine hydrochloride, and mitomycin C;
bortezomib, decitabine, and mitomycin C;
bortezomib, mechlorethamine hydrochloride, and decitabine; and
bortezomib, mechlorethamine hydrochloride, decitabine, and mitomycin C.

5. A method of treating bortezomib-resistant multiple myeloma in a subject in need thereof, comprising:
administering to the subject a pharmaceutically effective amount of each drug in a drug combination comprising bortezomib and at least one additional drug selected from the group consisting of mechlorethamine hydrochloride, decitabine, and mitomycin C.

6. A method of treating bortezomib-sensitive multiple myeloma in a subject in need thereof, comprising:
administering to the subject a pharmaceutically effective amount of each drug in a drug combination selected from the group consisting of:
bortezomib and dexamethasone;
bortezomib, panobinostat, and dexamethasone;
bortezomib, mechloroethamine hydrochloride, and dexamethasone; and
bortezomib, mechloroethamine hydrochloride, panobinostat, and dexamethasone.

7. The method of claim 4, wherein two or more drugs in the drug combination are administered sequentially.

8. The method of claim 4, wherein two or more drugs in the drug combination are administered concurrently.

9. The method of claim 4, wherein the subject is a mammal, optionally wherein the mammal is a human.

10. The method of claim 5, wherein the subject is a mammal, optionally wherein the mammal is a human.

11. The method of claim 4, wherein the pharmaceutically effective amount of each drug in the drug combination is below a maximum tolerated dosage.

12. The pharmaceutical composition of claim 2, wherein the pharmaceutically effective amount of each drug in the drug combination is below a maximum tolerated dosage.

13. The method of claim 5, wherein two or more drugs in the drug combination are administered sequentially.

14. The method of claim 6, wherein two or more drugs in the drug combination are administered sequentially.

15. The method of claim 5, wherein two or more drugs in the drug combination are administered concurrently.

16. The method of claim 6, wherein two or more drugs in the drug combination are administered concurrently.

17. The method of claim 6, wherein the subject is a mammal, optionally wherein the mammal is a human.

18. The method of claim 5, wherein the pharmaceutically effective amount of each drug in the drug combination is below a maximum tolerated dosage.

19. The method of claim 6, wherein the pharmaceutically effective amount of each drug in the drug combination is below a maximum tolerated dosage.
